(19) United States
(12) Patent Application Publication
(10) Pub. No.: US 2015/0212096 A1
(43) Pub. Date: Jul. 30, 2015

(54) Diet Support Device, Method, and Recording Medium Stored with Program

(71) Applicant: TANITA CORPORATION, Tokyo (JP)

(72) Inventors: Miyuki Kodama, Tokyo (JP); Ayumi Sano, Tokyo (JP); Naotaka Minagawa, Tokyo (JP); Yasuhiro Kasahara, Tokyo (JP)

(73) Assignee: TANITA CORPORATION, Tokyo (JP)

(21) Appl. No.: 14/602,914

(22) Filed: Jan. 22, 2015

(30) Foreign Application Priority Data
Jan. 27, 2014 (JP) 2014-012527

(51) Int. Cl. G01N 33/64; G01N 33/497; G01N 33/00

(52) U.S. Cl. CPC G01N 33/64; G01N 33/0047; G01N 33/497

(57) ABSTRACT

The invention provides a diet support device, method, and program capable of supporting healthy dieting.

A diet support device 10 acquires a ketone concentration measurement measuring ketone excreted from a user, determines the presence or absence of a rebound trend from a diet based on the acquired ketone concentration measurement, and outputs a determination result.

### CROSS-REFERENCE TO RELATED APPLICATION

This application is based on and claims the benefit of priority of the prior Japanese Patent Application No. 2014-012527, filed on Jan. 27, 2014, the entire contents of which are incorporated herein by reference.

### BACKGROUND

1. Technical Field

The present invention relates to a diet support device, method, and program.

2. Related Art

Hitherto, health management systems promoting improved lifestyles relating to diet and exercise have been proposed.

For example, Patent Document 1 describes technology to determine a dietary intake amount based on an activity level and an obesity level, and calculate and suggest a daily energy amount by which meals should be reduced, and a daily energy amount that should be reduced through exercise.

Patent Document 1: Japanese Patent Application Laid-Open (JP-A) No. 2008-250967

Although diets sometimes fail by rebounding when attempting to diet by improved living, such as diet and exercise, for example, the above conventional technology merely suggests energy amounts to be reduced through diet or exercise, making it hard to envisage what sort of diet or exercise should be performed to overcome a rebound and enable appropriate dieting. There is therefore an issue of struggling to make dietary and exercise improvements, and being unable to quickly overcome the rebound, leading to diets failing is many cases.

### SUMMARY

An object of the present invention is to provide a diet support device, method, and program capable of supporting healthy dieting.

In order to address the above issues, a diet support device of the present invention includes a ketone concentration acquisition section that acquires a ketone concentration measurement measuring ketone excreted from a user, a determination section that determines the presence or absence of a rebound trend from a diet based on the ketone concentration measurement acquired by the ketone concentration acquisition section, and an output section that outputs a determination result by the determination section.

Configuration may be made wherein the determination section determines the presence or absence of the rebound trend based on a comparison result between a variation curve expressing variation in the ketone concentration measurements, and based on a predetermined rebound curve expressing variation in the ketone concentration measurements when a rebound from a diet has occurred.

Configuration may be made further including a guidance energy amount acquisition section that, based on the ketone concentration measurement, acquires a guidance energy amount including at least one out of a guidance sugar amount to guide the user or a guidance exercise amount to guide the user.

Configuration may be made wherein the guidance energy amount acquisition section acquires the guidance energy amount based on the presence or absence of the rebound trend determined by the determination section, and the ketone concentration measurement.

Configuration may be made wherein the guidance energy amount acquisition section acquires the guidance energy amount based on at least one piece of user data selected from out of the group consisting of gender, age, build, and body composition of the user, and the ketone concentration measurement.

Configuration may be made wherein the guidance energy amount acquisition section acquires the guidance energy amount based on the difference between the ketone concentration measurement, and a target ketone concentration based on a target body weight and a target timeframe for the diet of the user.

Configuration may be made further including a menu acquisition section that acquires at least one out of a meal menu corresponding to the guidance energy amount or an exercise menu corresponding to the guidance energy amount.

Configuration may be made further including a measurement section that measures ketone excreted from the user.

The ketone excreted from the user may be acetone contained in breath exhaled from the user.

A diet support method of the present invention includes acquiring a ketone concentration measurement measuring ketone excreted from a user, determining the presence or absence of a rebound trend from a diet based on the acquired ketone concentration measurement, and outputting a determination result.

A diet support program of the present invention causes a computer to execute processing, the processing including acquiring a ketone concentration measurement measuring ketone excreted from a user, determining the presence or absence of a rebound trend from a diet based on the acquired ketone concentration measurement, and outputting a determination result.

### Advantageous Effects of Invention

The present invention exhibits the advantageous effect of enabling support of healthy dieting.

### BRIEF DESCRIPTION OF DRAWINGS

FIG. 1 is a diagram of the external appearance of a diet support device.

FIG. 2 is a block diagram of a diet support device.

FIG. 3 is a flowchart of processing by a diet support program.

FIG. 4 is a graph illustrating an example of a variation curve of acetone concentration, showing a case in which acetone concentration falls due to a rebound.

FIG. 5 is a drawing illustrating an example of a data table including ages, differences between measured acetone concentrations and target acetone concentrations, guidance sugar amounts, and guidance exercise amounts.

FIG. 6 is a drawing illustrating an example of a data table showing correspondence relationships between total energy amounts, meal menus, and exercise menus.

FIG. 7 is a block diagram illustrating a connection between a measurement instrument and a personal computer.

### DESCRIPTION OF EMBODIMENTS

Explanation follows regarding an exemplary embodiment of the present invention.

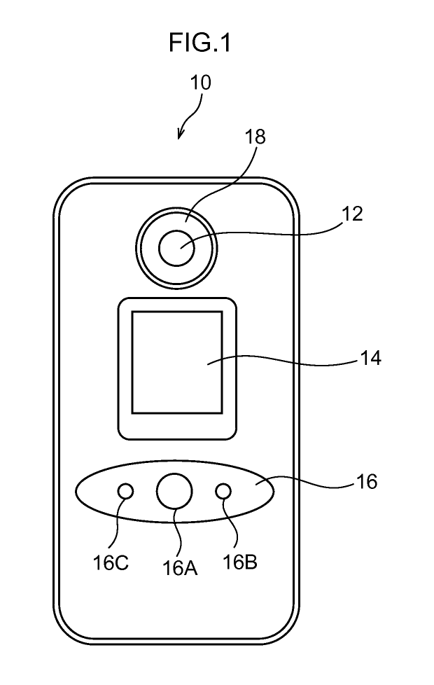

FIG. 1 is a diagram of the external appearance of a diet support device 10 according to the present exemplary embodiment. As illustrated in FIG. 1, the diet support device 10 includes a measurement section 12, a display section 14, and an operation section 16. The diet support device 10 according to the present exemplary embodiment is, as an example, a portable device that is convenient to be carried around.

The measurement section 12 measures the concentration of ketone excreted from a user (referred to below as ketone concentration). Ketone is a collective name employed for acetoacetic acid, 3-hydroxy acetic acid (β-hydroxy acetic acid), and acetone, and represents at least one thereof.

In the present exemplary embodiment, explanation is given of a case in which the measurement section 12 is, as an example, configured with an acetone detection sensor that detects acetone in the breath of a user. The user is able to measure the acetone concentration of exhaled breath by blowing air into a blow-in hole 18. In order to facilitate collection of exhaled air, the blow-in hole 18 may be a mouthpiece type shaped so as to be capable of being held in the mouth, or may be a mask type shaped as a mask.

The display section 14 is configured, for example, by a liquid crystal panel or the like. Various screen images are displayed on the display section 14, such as various setting screen images, the measurement results of acetone concentration measured by the measurement section 12, and advice information based on the measured acetone concentration. The display section 14 may also be configured including a touch panel function, and may be configured to enable operation by directly touching the screen image.

The operation section 16 is configured including plural operation buttons, and FIG. 1 illustrates an example of a case in which there are 3 individual operation buttons 16A to 16C provided.

The operation button 16A functions, as an example, as a button to operate to switch the power source of the diet support device 10 ON/OFF, and to make selections on various screen images.

The operation button 16B functions, as an example, as a button to input data on various screen images.

The operation button 16C functions, as an example, as a button to instruct reading of past measurement results and the like.

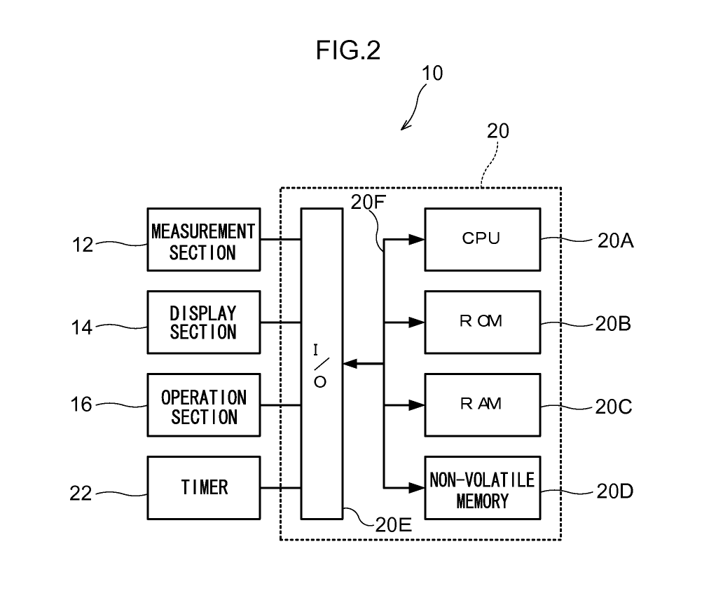

FIG. 2 is a block diagram of the diet support device 10. As illustrated in FIG. 2, the diet support device 10 includes a controller 20. The controller 20 is configured including a Central Processing Unit (CPU) 20A, Read Only Memory (ROM) 20B, Random Access Memory (RAM) 20C, non-volatile memory 20D, and an input-output (I/O) interface 20E, each connected together through a bus 20F. In this case, a diet support program that causes the CPU 20A of the controller 20 to execute diet support processing, explained later, is, for example, pre-written to the non-volatile memory 20D, and read into and executed by the CPU 20A. The diet support program may be provided on a recording medium, such as a CD-ROM, memory card, or the like, or may be downloaded from a server, not illustrated in the drawings.

The measurement section 12, the display section 14, the operation section 16, and a timer 22 are connected to the I/O interface 20E. The timer 22 includes a function to acquire the current time, and a timing function to time a set duration.

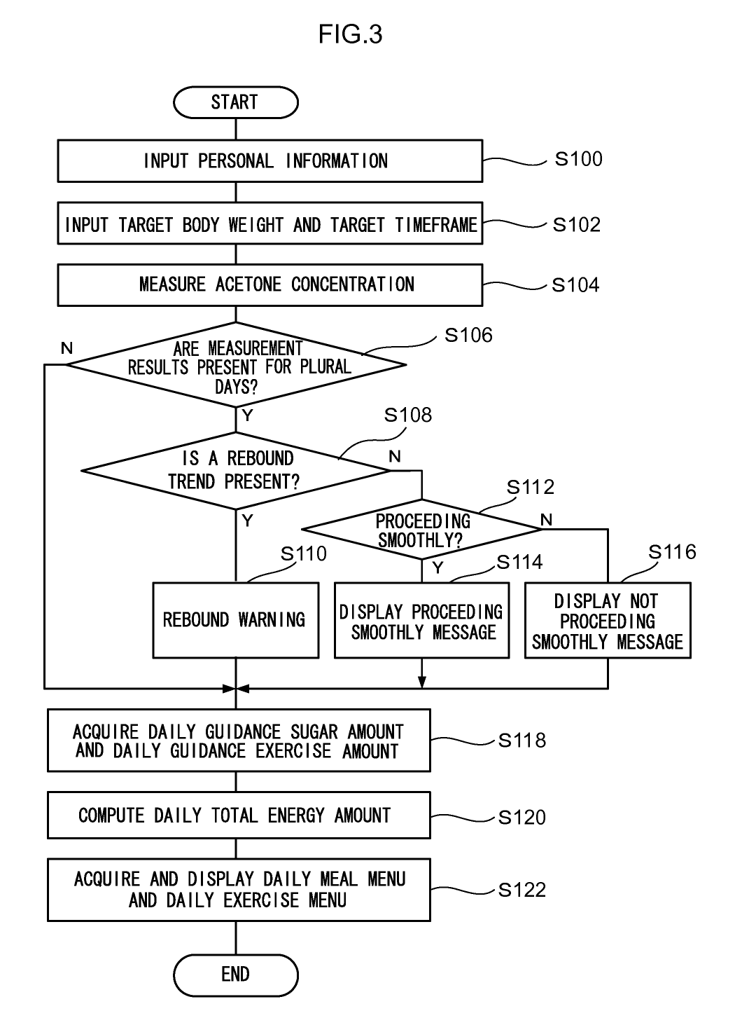

Explanation next follows, as operation of the present exemplary embodiment, regarding processing by the diet support program executed by the CPU 20A of the controller 20, with reference to the flowchart illustrated in FIG. 3. The processing in FIG. 3 is executed when a user operates the operation section 16 of the diet support device 10, and instructs execution of the diet support program.

At step S100, a personal information input screen image is displayed on the display section 14, and the user inputs their personal information. The personal information includes at least one out of, for example, gender, age, build data, or body composition data. The build data may, for example, be body weight, or may be data indicating builds, such as “underweight”, “standard”, or “obese”. Moreover, body weight and height may be input and Body Mass Index (BMI) calculated, with the calculated BMI being used as the build data. The body composition data may be, for example, body fat percentage, visceral fat level, or muscle mass, but is not limited thereto. The personal information input by the user is stored in the non-volatile memory 20D.

At step S102, the display section 14 displays a target input screen image for input of a target body weight and input of a target timeframe in which to achieve the target body weight, and the user inputs the target body weight and the target timeframe. The target body weight and the target timeframe input by the user are stored in the non-volatile memory 20D.

At step S104, acetone concentration is measured. More specifically, first a message to start measuring acetone concentration is displayed on the display section 14 after a predetermined time (for example 10 seconds) has elapsed, and the timer 22 is instructed to time a predetermined duration.

Then when notified by the timer 22 that the predetermined duration has elapsed, a blow-in start message instructing air to be exhaled through the blow-in hole 18 is displayed on the display section 14, and the measurement section 12 is instructed to start measuring the acetone concentration. The user exhales air into the blow-in hole 18 when the blow-in start message is displayed on the display section 14.

The measurement section 12 measures the acetone concentration of the air exhaled into the blow-in hole 18 and outputs the measurement to the controller 20. The measured acetone concentration is stored in the non-volatile memory 20D together with the current time acquired from the timer 22.

At step S106, determination is made as to whether or not measurement results of acetone concentration over plural days, including the day of the current measurement, are stored in the non-volatile memory 20D. Processing transitions to step S108 if stored, and processing transitions to step S118 if not stored.

At step S108, determination is made as to whether or not a rebound trend is present based on the measurement results of acetone concentration over plural days. When the acetone concentration is measured plural times during the same day, the average value of all the acetone concentrations measured within the same day may, for example, be taken as the acetone concentration for that day.

The acetone concentration of acetone that is a byproduct of fat metabolism may be thought of as corresponding to the fat burn amount. The acetone concentration is lower when there is surplus sugar energy present in the body, and the acetone concentration is higher when there is not enough sugar energy present in the body. Accordingly, in cases in which a diet is progressing smoothly, the fat burn amount gradually increases, giving a gradual rise in acetone concentration as the days pass. However, when a rebound occurs from a progressing state of a diet, due to the rebound the acetone concentration falls rapidly from the high acetone concentration state, after which a low acetone concentration state is maintained for a specific period of time.

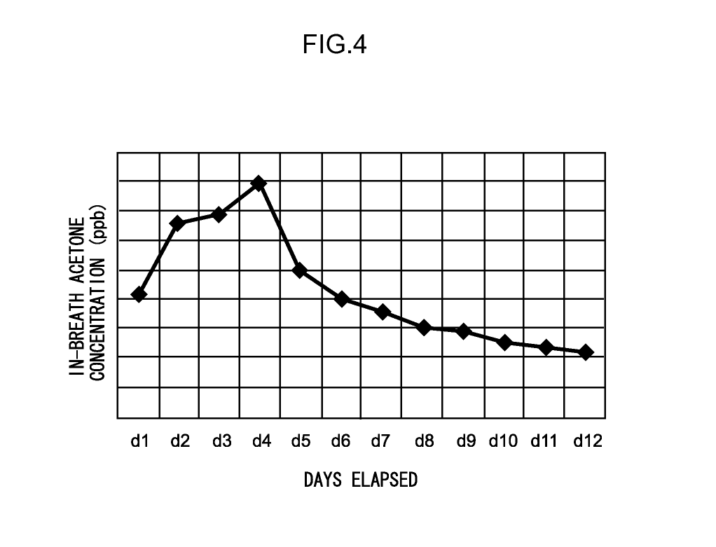

FIG. 4 illustrates an example of an acetone concentration variation curve when rebound has occurred. In the example illustrated in FIG. 4, a high acetone concentration state, namely a state in which there is not enough sugar energy in the body, continues from a day d1 to a day d4, and the high acetone concentration state continuing during this period may be thought of as being due to dieting. After the acetone concentration then falls sharply from day d4 to day d5, the acetone concentration then gradually decreases, with a low acetone concentration state maintained. The low acetone concentration state being maintained may be thought of as being due to excessive eating as a result of rebound and a low level of exercise. Namely, the acetone concentration variation curve when rebound occurs is a curve that falls rapidly after a high acetone concentration state has continued for a specific period of time, and then maintains a low acetone concentration state for another specific period of time.

Determination is made in such cases as to whether or not a rebound trend can be observed by determining whether or not the acetone concentration variation curve (referred to below as the measurement curve) measured over plural days resembles at least part of a curve (referred to below as a rebound curve) that falls rapidly after continuing at a high acetone concentration state for a specific period of time, and then maintains a low acetone concentration state for a specific period of time.

Whether or not there has been a sharp fall is determined by determining a sharp fall when (A2−A1)/(D2−D1) is less than a predetermined first threshold value, wherein A1 is the acetone concentration at a start day D1 of a period for determining whether or not there is a sharp fall, and A2 is the acetone concentration at the final day D2 thereof. Namely, a sharp fall is determined when the slope of a line connecting the acetone concentration of the start day of the period for determining whether or not there is a sharp fall and the acetone concentration at the final day thereof is less than the predetermined first threshold value. Determination of a sharp fall may also be made when the difference (A2−A1) between the acetone concentration A1 at the start day D1 of the period for determining whether or not there is a sharp fall, and the acetone concentration A2 at the final day D2 thereof, is less than a predetermined second threshold value, or determination of a sharp fall may also be made when a ratio (A2/A1) is less than a predetermined third threshold value.

Moreover, for example, the similarity between the measurement curve and at least part of the rebound curve may also be calculated using a known method, and a rebound trend determined to have been observed when the calculated similarity is a predetermined fourth threshold value or greater.

A rebound trend may be determined to be present not only when the measurement curve resembles the entire rebound curve, but also when the measurement curve resembles part of the rebound curve. For example, in the rebound curve illustrated in FIG. 4, the high acetone concentration state continues for the specific period of time. When such a state persists for a long time in the measurement curve, determination may be made that there is a possibility of a subsequent rebound occurring with a low acetone concentration then continuing, and determination of a rebound alert may be made.

Processing transitions to step S110 when a rebound trend is determined to have been observed at step S108, and processing transitions to step S112 when a rebound trend is determined not to have been observed.

At step S110, a message warning that a rebound trend has been observed is displayed on the display section 14. This thereby enables a user to be made aware of the rebound trend, enabling the user to take care to diet healthily and suppress a rebound from occurring.

At step S112, determination is made as to whether or not the diet is proceeding smoothly based on the acetone concentrations measurement results over plural days. The acetone concentration gradually increases when the diet is proceeding smoothly. Determination of a rising acetone concentration be made, for example, when (A4−A3)/(D4−D3) is a predetermined fourth threshold value or greater, wherein A3 is the acetone concentration at a start day D3 of a period for determining whether or not the acetone concentration is rising, and A4 is the acetone concentration at the final day D4 thereof. Namely, a rising acetone concentration is determined when the slope of a line connecting the acetone concentration of the start day of the period for determining whether or not there is a rising acetone concentration and the acetone concentration at the final day thereof is the predetermined fourth threshold value or greater. Determination of a rising acetone concentration may also be made when the difference (A4−A3) between the acetone concentration A3 at the start day D3 of the period for determining whether or not there is a rising acetone concentration, and the acetone concentration A4 at the final day D4 thereof is a predetermined fifth threshold value or greater, or determination of a rising acetone concentration may by made when a ratio (A4/A3) is a predetermined sixth threshold value or greater.

Each of the threshold values described above may, for example, be set according to the difference between the target body weight and the current body weight. For example, the fourth threshold value may become smaller the smaller the difference to the target body weight. This increases the possibility of determining the diet is proceeding smoothly as the target body weight is approached.

Processing transitions to step S114 when the diet has been determined to be proceeding smoothly, and processing transitions to step S116 when the diet has not been determined to be proceeding smoothly.

At step S114, a message showing that the diet is proceeding smoothly is displayed on the display section 14. However, step S116, a message that the diet is not proceeding smoothly is displayed on the display section 14.

At step S118, a guidance energy amount, including at least one out of a daily guidance sugar amount or guidance exercise amount, is acquired as guidance for the user, based on the acetone concentration measured at step S104. The guidance sugar amount encompasses both amounts of sugar that should be consumed, or amounts by which sugar should be cut.

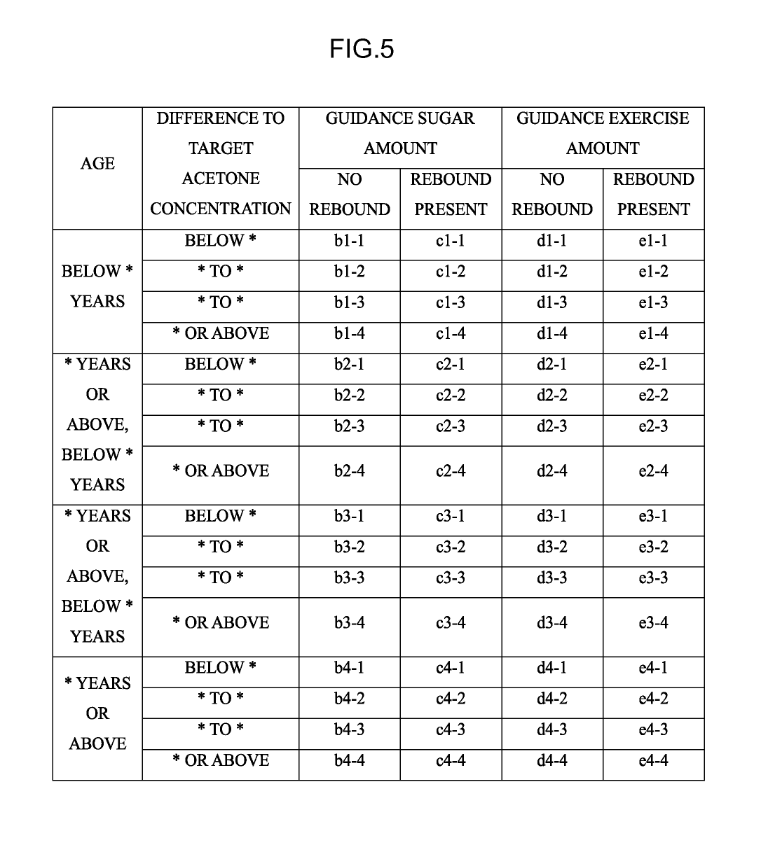

The guidance sugar amount and the guidance exercise amount are retrieved from a data table such as that illustrated in FIG. 5. The data table illustrated in FIG. 5 is a data table stored in the non-volatile memory 20D, and is preset with guidance sugar amounts and guidance exercise amounts according to age, and the difference between current acetone concentrations and target acetone concentrations. Columns for the guidance sugar amounts and guidance exercise amounts are preset with values in units such as kilocalories (kcal). The guidance sugar amounts and guidance exercise amounts are respectively set for whether or not a rebound trend is present. Namely, if a rebound trend has been determined to be present at step S108, the guidance sugar amount and guidance exercise amount are retrieved from the “rebound present” columns, and if a rebound trend has been determined not to be present at step S108, or if determination was made at step S106 that acetone concentrations measurement results over plural days are not present, namely if the acetone concentration has been determined just once, the guidance sugar amount and guidance exercise amount are retrieved from the “rebound absent” columns.

Accordingly, at step S118, the guidance sugar amount and guidance exercise amount corresponding to the age of the user input at step S100, the difference between the acetone concentration measured at step S104 and the target acetone concentration, and the presence or absence of a rebound trend, are acquired from the data table illustrated in FIG. 5.

The target acetone concentration is calculated based on the target body weight and the target timeframe input at step S102. For example, the target acetone concentration is calculated using a predetermined calculation formula for computing a target acetone concentration using parameters of the target body weight and the target timeframe.

The data table illustrated in FIG. 5 includes both the guidance sugar amounts and the guidance exercise amounts, but it is enough if the data table includes either one of them. The guidance sugar amount and guidance exercise amount may also be derived directly from the acetone concentration measured at step S104. In such cases, for example a data table indicating correspondence relationships between acetone concentration, guidance sugar amount, and guidance exercise amount may be stored in advance in the non-volatile memory 20D, and the data table may be used to derive the guidance sugar amount and guidance exercise amount corresponding to the measured acetone concentration.

The data table illustrated in FIG. 5 may also be categorized by gender, build, and body composition to retrieve the guidance sugar amount and guidance exercise amount according to gender, build, and body composition.

At step S120, a daily total energy amount that should be consumed or cut henceforth is calculated using for example a predetermined calculation formula, based on the guidance sugar amount retrieved at step S118. The calculation formula may, for example, be a formula to calculate a total energy amount in order to achieve an appropriate energy ratio between the three primary macronutrients of protein, fat, and carbohydrates, namely an appropriate PFC balance.

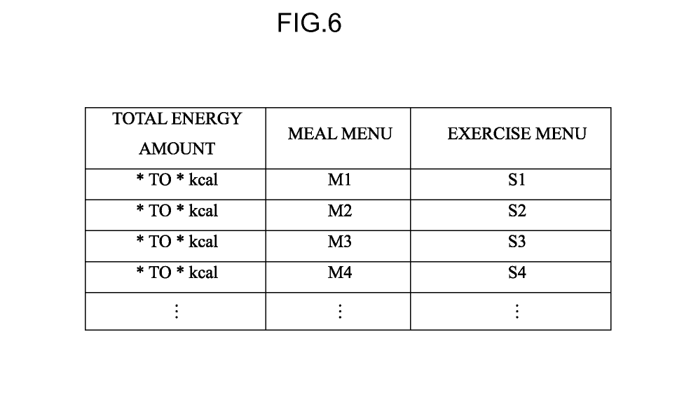

At step S122, a meal menu and exercise menu are retrieved corresponding to the total energy amount calculated at step S120, and are displayed on the display section 14. For example, a data table such as that illustrated in FIG. 6, indicating correspondence relationships between total energy amounts, meal menus, and exercise menus is employed to retrieve the meal menu and the exercise menu corresponding to the total energy amount, and the retrieved meal menu and exercise menu are displayed on the display section 14. The total energy amount column in the data table in FIG. 6 is preset with values in units such as kilocalories (kcal), the meal menu column is preset with text indicating meal menus, and the exercise menu column is preset with text indicating exercise menus.

Displaying the meal menu and exercise menu in place of the required total energy amount enables easy comprehension of specifically what sort of meals and exercise should be taken in order to enable dieting. Configuration may alternatively be made to retrieve and display only the meal menu corresponding to the total energy amount, or to retrieve and display only the exercise menu corresponding to the total energy amount.

Explanation has been given regarding a case in which one meal menu and one exercise menu corresponding to the total energy amount are displayed one at a time; but the data table illustrated in FIG. 6 may be preset with plural meal menus and plural exercise menus corresponding to the total energy amounts, and plural meal menus and exercise menus corresponding to the total energy amount may be displayed in one go. This thereby enables the user to choose their preferred menus.

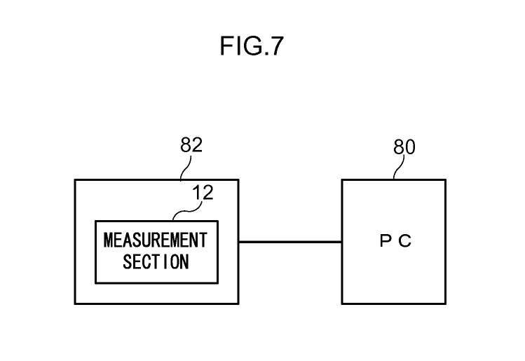

Although explanation has been given in the present exemplary embodiment regarding a case in which the diet support device 10 is a dedicated portable device, configuration may be made in which a measurement instrument 82 including a measurement section 12 is connected by wire, or wirelessly, to a personal computer 80, as illustrated in FIG. 7. In such cases the personal computer 80 functions as a diet support device by acquiring an acetone concentration measured by the measurement instrument 82, and executing the processing illustrated in FIG. 3.

The device connected to the measurement instrument 82 is not limited to a personal computer, and may be a portable terminal such as a mobile phone, a smart phone, or a tablet terminal. The measurement section 12 may also be built into such a portable terminal. Configuration may be made such that a portable terminal or the measurement instrument 82 is connected to a server over a network. In such cases the server functions as the diet support device. Namely, the portable terminal or the measurement instrument 82 transmits the measured acetone concentration to a server, the server then executes the processing illustrated in FIG. 3 based on the acetone concentration received from the portable terminal or the measurement instrument 82, and transmits the result to the portable terminal or the measurement instrument 82. The measurement instrument 82 may thereby be provided with minimum functionality of measuring and transmitting the acetone concentration to the server, and functionality to receive the result from the server and display, enabling a cost effective configuration to be achieved.

Explanation has been given in the present exemplary embodiment regarding a case in which the measurement section 12 is configured including an acetone detection sensor that detects in-breath acetone, but there is no limitation thereto, and configuration may be made in which a ketone detection sensor is provided that detects excreted ketone, such as in the skin, urine, saliva, or sweat of a user.

What is claimed is:

1. A diet support device comprising:
a ketone concentration acquisition section that acquires a ketone concentration measurement measuring ketone excreted from a user;
a determination section that determines the presence or absence of a rebound trend from a diet based on the ketone concentration measurement acquired by the ketone concentration acquisition section; and
an output section that outputs a determination result by the determination section.

2. The diet support device of claim 1, wherein
the determination section determines the presence or absence of the rebound trend based on a comparison result between a variation curve expressing variation in the ketone concentration measurements, and based on a predetermined rebound curve expressing variation in the ketone concentration measurements when a rebound from a diet has occurred.

3. The diet support device of claim 1, further comprising
a guidance energy amount acquisition section that, based on the ketone concentration measurement, acquires a guidance energy amount including at least one out of a guidance sugar amount to guide the user or a guidance exercise amount to guide the user.

4. The diet support device of claim 3, wherein
the guidance energy amount acquisition section acquires the guidance energy amount based on the presence or absence of the rebound trend determined by the determination section, and the ketone concentration measurement.

5. The diet support device of claim 3, wherein
the guidance energy amount acquisition section acquires the guidance energy amount based on at least one piece of user data selected from the group consisting of gender, age, build, and body composition of the user, and the ketone concentration measurement.

6. The diet support device of claim 3, wherein
the guidance energy amount acquisition section acquires the guidance energy amount based on the difference between the ketone concentration measurement, and a target ketone concentration based on a target body weight and a target timeframe for the diet of the user.

7. The diet support device of claim 3, further comprising a menu acquisition section that acquires at least one out of a meal menu corresponding to the guidance energy amount or an exercise menu corresponding to the guidance energy amount.

8. The diet support device of claim 1, further comprising a measurement section that measures ketone excreted from the user.

9. The diet support device of claim 1, wherein the ketone excreted from the user are acetone contained in breath exhaled from the user.

10. A diet support method comprising:
acquiring a ketone concentration measurement measuring ketone excreted from a user;
determining the presence or absence of a rebound trend from a diet based on the acquired ketone concentration measurement; and
outputting a determination result.

11. A non-transitory recording medium stored with a diet support program that causes processing to be executed on a computer, the processing comprising:
acquiring a ketone concentration measurement measuring ketone excreted from a user;
determining the presence or absence of a rebound trend from a diet based on the acquired ketone concentration measurement; and
outputting a determination result.
